(19) United States
(12) Patent Application Publication
(10) Pub. No.: US 2025/0221907 A1
(43) Pub. Date: Jul. 10, 2025

(54) COSMETIC COMPOSITION COMPRISING BIS-ETHYLHEXYLOXYPHENOL METHOXYPHENYL TRIAZINE

(71) Applicant: BASF SE, Ludwigshafen am Rhein (DE)

(72) Inventors: Myriam SOHN, Grenzach-Wyhlen (DE); Stanislaw KRUS, Grenzach-Wyhlen (DE)

(21) Appl. No.: 18/404,221

(22) Filed: Jan. 4, 2024

(51) Int. Cl. A61K 8/49; A61K 8/27; A61K 8/29; A61K 8/40; A61K 8/81; A61Q 17/04

(52) U.S. Cl. CPC A61K 8/4966; A61K 8/27; A61K 8/29; A61K 8/40; A61K 8/8158; A61Q 17/04; A61K 2800/30; A61K 2800/592

(57) ABSTRACT

Cosmetic composition comprising
(i) bemotrizinol (bis-ethylhexyloxyphenol methoxyphenyl triazine),
(ii) a water-soluble organic UV filter or a salt thereof,
(iii) at least one inorganic UV filter,
wherein the cosmetic composition is free of the UV filters from the group consisting of hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), polysilicone-15, and 2-hydroxy-4-methoxybenzophenone (INCI benzophenone-3) and a method for increasing the water resistance of a cosmetic composition comprising bis-ethylhexyloxyphenol methoxyphenyl triazine (bemotrizinol), zinc oxide, and titanium dioxide comprising the step of adding a water-soluble organic UV filter or a salt thereof to the cosmetic composition.

### TECHNICAL FIELD

The present invention relates to a cosmetic composition comprising (i) bemotrizinol (INCI: bis-ethylhexyloxyphenol methoxyphenyl triazine), (ii) a water-soluble organic UV filter or a salt thereof, and (iii) at least one inorganic UV filter.

### BACKGROUND

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filters, UV-B filters and broadband filters. These filters are added to sunscreen or daily care compositions. The UV filters are either organic or inorganic, particulate or non-particulate compounds, of which all have a high absorption efficacy in the UV-light range. In general, UV light radiation relevant for sun protection products can be divided into UV-A radiation and UV-B radiation. Depending on the position of the absorption maxima, UV-filters are divided into UV-A and UV-B filters. In case a UV-filter absorbs both, UV-A and UV-B light, it is referred to as a broadband absorber. Since broadband UV filters provide protection against UV-A and UV-B radiation it is preferred to add these kind of UV filters into sunscreen or daily care composition to ensure a broad and efficient UV protection foundation over the whole UV range.

Generally, sunscreen compositions comprise a multitude of UV filters and further components to provide optimal protection across the entire UV-A and UV-B range. However, this approach can come with several disadvantages. For one, the sourcing of a complex mixture of different UV filters can be daunting and expensive, thus also driving up the price of the final product. Additionally, specific UV filter combinations might result in photounstable formulations or components, for example the combination of the UVA filter avobenzone and the UVB filter octinoxate has been found to result in the photodecomposition of avobenzone and further in a hetero-cycloaddition of both molecules resulting in a significant loss of UV protection. Finally, some of the most commonly used UV filters are under suspicion for their health and environmental risks. For example, the commonly used UV filters octocrylene, homosalate, octinoxate, and oxybenzone are under suspicion to exhibit hormone-like behavior in the human body.

Therefore, it is desirable to develop cosmetic compositions that can provide excellent protection from UV radiation and have excellent overall properties regarding skin feel, glossiness and stickiness while requiring as few different UV filters as possible.

It is thus an objective of the present invention to provide a composition that can be used for sunscreen products with sun protection levels spanning from low to high SPF (SPF from 6 to more than 60), low glossiness, and low stickiness while requiring a small number of different UV filters.

There are only few UV filters that have the ability to cover the entire range of harmful UV light irradiation without any known negative impact on the environment or human health. Bemotrizinol (INCI: Bis-ethylhexyloxyphenol methoxyphenyl triazine; abbreviation: BEMT, compound (1)) stands out in this group of broadband UV filters since it exhibits a wide and high sun protection band in the UV-A and UV-B range with a E1,1 (extinction 1% filter at an optical thickness of 1 cm) of >730 in the UVB and higher than 800 in the UVA range while having a strong photostability and a good health and environmental profile.

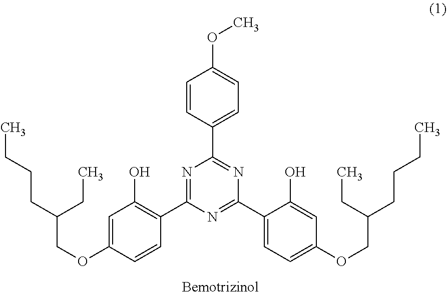 [SMILES: CCCCC(CC)COc1ccc(-c2nc(-c3ccc(OC)cc3)nc(-c3ccc(OCC(CC)CCCC)cc3O)n2)c(O)c1]

The combination of bemotrizinol with the inorganic UV filters zinc oxide and titanium dioxide provides a highly effective sunscreen composition that provides excellent UV protection across the entire relevant UV light range. In addition, these three UV filters are all considered ecologically and environmentally benign as well as particularly safe for use. Furthermore, zinc oxide and titanium dioxide are currently the only UV filters considered category 1 GRASE (generally recognized as safe and effective) by the FDA. Bemotrizinol could join these two UV filters as the third category 1 GRASE UV filter.

Unfortunately, however, the combination of these three UV filters in cosmetic compositions has been found to have poor water resistance. Since sunscreen compositions are particularly relevant for the use during hot weather on the beach or near a swimming pool, a poor water resistance is a major obstacle for the use of a sunscreen composition.

Therefore, there is a need to develop cosmetic compositions comprising the UV filter combination of bemotrizinol with one or more inorganic UV filters with improved water resistance.

### SUMMARY OF THE INVENTION

Surprisingly, it was now discovered that the water resistance of such a cosmetic composition can be significantly increased by the addition of a water-soluble organic UV filter or a salt thereof.

In a first aspect, the invention relates to a cosmetic composition comprising
(i) bemotrizinol (INCI: bis-ethylhexyloxyphenol methoxyphenyl triazine),
(ii) a water-soluble organic UV filter or a salt thereof,
(iii) at least one inorganic UV filter,
wherein the cosmetic composition is free of the UV filters from the group consisting of hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), polysilicone-15, and 2-hydroxy-4-methoxybenzophenone (INCI benzophenone-3). It was discovered that such a cosmetic composition provides excellent UV protection across the entire harmful UV spectrum while simultaneously providing increased water resistance compared to a similar composition that does not comprise the water-soluble organic UV filter or a salt thereof. It is particularly surprising that a water-soluble UV filter can help for water resistance.

In a second aspect, the invention relates to a method for increasing the water resistance of a cosmetic composition comprising bemotrizinol, zinc oxide, and titanium dioxide comprising the step of adding a water-soluble organic UV filter or a salt thereof to the cosmetic composition.

The present invention will be described with respect to particular embodiments and with reference to certain examples, but the invention is not limited thereto, and it is only defined by the appending claims.

### Definitions

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±15%, preferably ±10%, and more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

As used herein the term “does not comprise” or “free of” means in the context that the composition of the present invention is free of a specific compound or group of compounds, which may be combined under a collective term, that the composition does not comprise said compound or group of compounds in an amount of more than 0.1% by weight, based on the total weight of the composition. Furthermore, it is preferred that the composition according to the present invention does not comprise said compounds or group of compounds in an amount of more than 0.05% by weight, preferably the composition does not comprise said compounds or group of compounds in any measurable quantity using standard analytical equipment. Furthermore, it is to be understood that the term “free of” or “does not comprise”, in case the composition does not comprise a compound X and a compound Y means that the compositions must be free of both compounds X and Y. In other words, the composition may not contain any compound selected from X and Y.

When referring to compositions and the weight percent of the therein comprised ingredients it is to be understood that according to the present invention the overall amount of ingredients does not exceed 100% (±0.1% due to rounding). If numerical values are given without any digits, the respective value comprises the range that arises from normal rounding. For example, a value of 10 thus comprises the range of from 9.5 to 10.4. The same applies if digits are given with respect to the next digit. For example, a value of 0.5 thus covers the range of from 0.45 to 0.54.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

Cosmetic compositions refer here to topical products. The term “topical product” refers to a product that is applied to the skin and can be a leave-on or rinse-off products. Preferred topical products of present invention are leave-on products. Suitable leave-on products of this invention can be intended to be used for face and/or for body.

Cosmetic compositions of present invention include sunscreens, or daily skin care compositions, or decorative preparations and can refer to a variety of formats such as for example sprays, lotions, creams, oils, gels, sticks, foams, powders, pastes, aerosols.

The term “sunscreen composition” refers to any topical product, which absorb and further may reflect certain parts of UV radiation. Thus, the term “sunscreen composition” is to be understood as not only including sunscreen compositions, but also any cosmetic compositions that provide UV protection.

The term “daily skin care composition” refers to any topical product which is intended to be used daily without or with a UV protection. In case UV protection is included, it absorbs and may further reflect and scatter certain parts of UV radiation.

Suitable decorative preparations refer to make up products and are, e.g., lipsticks, nail varnishes, eye shadows, mascaras, dry and moist make-up, rouge, powders, and suntan lotions.

The term “UV filter” or “ultraviolet filter” as used herein refers to organic or inorganic compounds, which can absorb and may further reflect and scatter UV radiation caused by sunlight. UV-filter can be classified based on their UV absorbance profile as UV-A, UV-B, or broadband filters. Preferably, the term “UV filter” comprises or consists of any UV filter as defined in the Annex VI (version of 11.11.2022) of the Regulation (EC) No 1223/2009 of the European Parliament and of the Council.

Water soluble UV filters have a solubility in water of at least 2.0% by weight, preferably at least 3.0% by weight, more preferably at least 5.0% by weight.

Oil soluble filters have a solubility in specific emollients of at least 2.0% by weight, preferably at least 3.0% by weight, more preferably at least 5.0% by weight.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. Alternatively, or additionally, the emollients improve the distribution of the product on the skin. Emollients can be divided into different groups depending on their polarity index.

The term “sun protection factor (SPF)” as used herein indicates how well the skin is protected by a sunscreen composition from an erythema (sunburn) induced mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm2).

The definition of “broadband” protection (also referred to as broad-spectrum or broad protection) may be based for example on the “critical wavelength” or the UVA1/UV ratio or the UVA-PF. For broadband protection, UV-B and UV-A protection must be provided. According to the US requirements, a critical wavelength of at least 370 nm is required for achieving broad spectrum protection. In the proposed order (OTCO000008) published Sep. 24, 2021, the FDA proposed to add an additional requirement to pass the broad-spectrum test, the product shall meet a UVA1/UV ratio of at least 0.7. Furthermore, it is recommended by the European Commission that all sunscreens or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at minimum at 370 nm.

The term “administration” refers to the application of a cosmetic composition to the skin of a person.

### Particular Embodiments of the Invention

The first aspect of the invention relates to a cosmetic composition comprising
(i) bemotrizinol (INCI: bis-ethylhexyloxyphenol methoxyphenyl triazine),
(ii) a water-soluble organic UV filter or a salt thereof,
(iii) at least one inorganic UV filter,
wherein the cosmetic composition is free of the UV filters from the group consisting of hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), polysilicone-15, and 2-hydroxy-4-methoxybenzophenone (INCI benzophenone-3). The inventive cosmetic composition shows a significantly improved water resistance determined by the contact angle measurement method compared to similar cosmetic compositions that do not comprise the water-soluble organic UV filter or salt thereof. In addition to this, the inventive cosmetic compositions only comprise environmentally and ecologically benign UV filters that are safe to use and pose no risk of hormonal imbalance.

According to one embodiment of the invention, the cosmetic composition is free of 2-ethylhexyl-2-cyano-3,3-diphenylprop-2-enoate (INCI octocrylene). Octocrylene is under suspicion to exhibit hormone-like behavior in the human body.

According to one embodiment of the invention, the cosmetic composition is free of 1-(4-(1,1-dimethylethyl)phenyl)-3-(4-methoxyphenyl)propane-1,3-dione (avobenzone). Under specific conditions, avobenzone can be unstable in combination with inorganic UV filters such as zinc oxide or titanium dioxide. For this reason, it is preferred that the cosmetic composition does not comprise avobenzone.

According to one embodiment of the invention, the cosmetic composition is free of (RS)-2-Ethylhexyl (2E)-3-(4-methoxyphenyl)prop-2-enoate (octinoxate). Octinoxate is also under suspicion to exhibit hormone-like behavior in the human body.

According to one embodiment of the invention, the cosmetic composition is free of 4,4′,4″-(1,3,5-triazine-2,4,6-triyltriimino)tris-benzoic acid-tris(2-ethylhexyl)ester (octyl triazone).

According to another embodiment of the invention, the cosmetic composition is free of any further organic UV filter. The UV filter combination of the inventive sunscreen composition does not require any further organic UV filter in order to provide excellent UV radiation protection. By limiting the number of different UV filters to the combination of bemotrizinol, one or more water-soluble organic UV filter or a salt thereof, and at least one inorganic UV filter, the risk of allergic skin reaction is greatly reduced.

The inventive cosmetic composition comprises at least one inorganic UV filter (III). According to one embodiment of the invention, the at least one inorganic UV filter (III) is selected from zinc oxide, titanium dioxide, and mixture thereof. According to one particularly preferred embodiment of the invention, the cosmetic composition comprises zinc oxide and titanium dioxide.

According to one embodiment of the invention, the water-soluble organic UV filter (ii) is selected from the group consisting of Phenyl-3H-benzimidazole-5-sulfonic acid (Ensulizole), [(3Z)-3-[[4-[(Z)-[7,7-Dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (Ecamsule), disodium phenyl dibenzimidazole tetrasulfonate (Bisdisulizole disodium), or their salts, and mixtures thereof. These water-soluble organic UV filters are well-suited to increase the water resistance of a cosmetic composition comprising bemotrizinol and at least one inorganic UV filter.

According to a preferred embodiment of the invention, the water-soluble organic UV filter (ii) is Phenyl-3H-benzimidazole-5-sulfonic acid (Ensulizole), or a salt thereof, and mixtures thereof. Ensulizole in the acid or salt form is particularly well-suited to increase the water resistance of a cosmetic composition comprising bemotrizinol and at least one inorganic UV filter.

According to a one embodiment, the cosmetic composition further comprises an organic neutralizing agent. It is further preferred that Ensulizole is used as a salt of an organic nitrogen compound. It is further preferred that the organic neutralizing agent is selected from tromethamine, triethanolamine, tetrahydroxypropyl ethylenediamine, and arginine, aminomethylpropanol.

According to one embodiment of the invention, the molar ratio of the water-soluble organic UV filter (ii) to the organic neutralizing agent is from 3:1 to 1:3, in particular from 2:1 to 1:2 or from 1.5:1 to 1:1.5. In one embodiment of the invention, the molar ratio of the water-soluble organic UV filter (ii) to the organic neutralizing agent is 1:1.

According to another preferred embodiment of the invention, the cosmetic composition comprises a Hydroxyethyl Acrylate/Sodium Acryloyldimethyl Taurate Copolymer (2-propenoic acid, 2-hydroxyethyl ester, polymer with 2-methyl-2-[(1-oxo-2-propenyl)amino]-1-propanesulfonic acid monosodium salt (CAS111286-86-3). Such polymers are known in the art and are sold for example under the tradename SEPINOV™ EMT 10 by Seppic. These polymers may be present in the described cosmetic compositions in an amount of from 0.3 to 3.0 wt. %, for example 0.5 to 2.0 wt. %, in particular 1.0 to 2.0 wt. %, based on the total weight of the cosmetic composition.

The inventive cosmetic composition comprises bemotrizinol. According to one embodiment of the invention, the cosmetic composition comprises bemotrizinol in an amount of from 0.5 to 6.0 wt. %, based on the total weight of the cosmetic composition. According to another embodiment of the invention, the cosmetic composition comprises bemotrizinol in an amount of from 0.5 to 5.5 wt. %, of from 1.0 to 5.0 wt. %, or from 1.5 to 5.0 wt. %, or from 2.0 to 5.0 wt. %, based on the total weight of the cosmetic composition. According to another embodiment of the invention, the cosmetic composition comprises bemotrizinol in an amount of from 2.0 to 4.0 wt. %, or of from 2.0 to 3.0 wt. %, or from 2.0 to 2.5 wt. %, based on the total weight of the cosmetic composition. The cosmetic composition described herein can, for example, comprise bemotrizinol in an amount of 0.5 wt. %, or of 1.0 wt. %, or of 1.5 wt. %, or of 2.0 wt. %, or of 2.5 wt. %, or of 3.0 wt. %, or of 3.5 wt. %, or of 4.0 wt. %, or of 4.5 wt. %, or of 5.0 wt. %, or of 5.5 wt. %, or of 6.0 wt. %, based on the total weight of the cosmetic composition.

The inventive cosmetic composition comprises a water-soluble organic UV filter or a salt thereof. According to one embodiment of the invention, the cosmetic composition comprises the water-soluble organic UV filter or salt thereof in an amount of from 0.1 to 4.0 wt. %, in particular of from 0.2 to 3.5 wt. %, or of from 0.5 to 3.0 wt. %, or from 1.0 to 2.5 wt. %, based on the total weight of the cosmetic composition. The cosmetic composition described herein can, for example, comprise the water-soluble organic UV filter or salt thereof in an amount of 0.5 wt. %, or of 1.0 wt. %, or of 1.5 wt. %, or of 2.0 wt. %, or of 2.5 wt. %, or of 3.0 wt. %, or of 3.5 wt. %, or of 4.0 wt. %, based on the total weight of the cosmetic composition.

A particularly well suited water-soluble organic UV filter is Ensulizole or a salt thereof. According to one embodiment of the invention, the cosmetic composition comprises Ensulizole or a salt thereof in an amount of from 0.1 to 4.0 wt. %, in particular of from 0.2 to 3.5 wt. %, or of from 0.5 to 3.0 wt. %, or from 1.0 to 2.5 wt. %, based on the total weight of the cosmetic composition. The cosmetic composition described herein can, for example, comprise Ensulizole or a salt thereof in an amount of 0.5 wt. %, or of 1.0 wt. %, or of 1.5 wt. %, or of 2.0 wt. %, or of 2.5 wt. %, or of 3.0 wt. %, or of 3.5 wt. %, or of 4.0 wt. %, based on the total weight of the cosmetic composition.

According to one embodiment of the invention, the cosmetic composition comprises zinc oxide.

If zinc oxide is present in the cosmetic composition, it is preferably that the zinc oxide is coated. Preferably, the zinc oxide is coated with inorganic and/or organic surface coatings. In a preferred embodiment of the present invention, the zinc oxide is coated with inorganic surface coating selected from the group consisting of sodium hexametaphosphate (NaPO3), sodium meta-phosphate (NaPO3)n, silicon dioxide (SiO2) (also: Silica, e.g. CAS-No.: 7631-86-9), iron oxide (Fe2O3), and mixtures thereof. It is to be understood that these inorganic surface coatings can be present on their own, in combination and/or in combination with organic surface coatings.

In a preferred embodiment of the present invention, the zinc oxide is coated with organic surface coatings selected from the group consisting of fatty acids such as stearic acid or lauric acid, dimethylpolysiloxane (also: dimethicone), methylpolysiloxane (methicone), simethicone (a mixture of dimethylpolysiloxane with an average chain length of about 200 to about 350 dimethylsiloxane units and silica gel), triethoxycaprylylsilane, octyltrimethoxysilane, and mixtures thereof. It is to be understood that these organic surface coatings can be present on their own, in combination and/or in combination with inorganic surface coatings. Preferably, the zinc oxide according to the present invention is uncoated or coated with dimethicone, methicone, or triethoxycaprylylsilane, most preferably the zinc oxide according to the present invention is surface coated with triethoxycaprylylsilane.

According to one embodiment of the invention, the cosmetic composition comprises zinc oxide in an amount of from 0.5 to 25.0 wt. %, or of from 0.5 to 20 wt. %, or of from 1.0 to 15.0 wt. %, or of from 1.5 to 10 wt. %, or of from 2.0 to 8.0 wt. %, or of from 2.5 to 6.0 wt. %, or from 3.5 to 5.5 wt. %, based on the total weight of the cosmetic composition. The cosmetic composition described herein can, for example, comprise zinc oxide in an amount of 0.5 wt. %, or of 1.0 wt. %, or of 1.5 wt. %, or of 2.0 wt. %, or of 2.5 wt. %, or of 3.0 wt. %, or of 3.5 wt. %, or of 4.0 wt. %, or of 4.5 wt. %, or of 5.0 wt. %, or of 5.5 wt. %, or of 6.0 wt. %, or of 6.5 wt. %, or of 7.0 wt. %, or of 7.5 wt. %, or of 8.0 wt. %, or of 8.5 wt. %, or of 9.0 wt. %, or of 9.5 wt. %, or of 10.0 wt. %, or of 10.5 wt. %, or of 11.0 wt. %, or of 11.5 wt. %, or of 12.0 wt. %, or of 12.5 wt. %, or of 13.0 wt. %, or of 13.5 wt. %, or of 14.0 wt. %, or of 14.5 wt. %, or of 15.0 wt. %, or of 15.5 wt. %, or of 16.0 wt. %, or of 16.5 wt. %, or of 17.0 wt. %, or of 17.5 wt. %, or of 18.0 wt. %, or of 18.5 wt. %, or of 19.0 wt. %, or of 19.5 wt. %, or of 20.0 wt. %, or of 20.5 wt. %, or of 21.0 wt. %, or of 21.5 wt. %, or of 22.0 wt. %, or of 22.5 wt. %, or of 23.0 wt. %, or of 23.5 wt. %, or of 24.0 wt. %, or of 24.5 wt. %, or of 25.0 wt. %, based on the total weight of the cosmetic composition.

According to one embodiment of the invention, the cosmetic composition comprises titanium dioxide.

In a preferred embodiment of the present invention, the titanium dioxide (iii) is a nano material. In this connection it is to be understood that the term “nano material” follows the recommendation of the European Commission 2011/696/EU. Accordingly, in a nano material 50% of particles, based on a number-based size distribution, are smaller than 100 nm, including constituent particles in aggregates or agglomerates. In another preferred embodiment of the present invention, the titanium dioxide is a non-nano material. Accordingly, in a non-nano material more than 50% of the particles, based on a number-based size distribution, are equal or larger than 100 nm.

In a preferred embodiment of the present invention, the titanium dioxide has a number-average elementary particle diameter of less than 1000 nm, preferably of less than 200 nm, more preferably of less than 100 nm, and in particular less than 50 nm. It is further preferred that the titanium dioxide has a number-average elementary particle diameter of 0.1 to less than 1000 nm, more preferably of 1 to 100 nm, and in particular of 2 to 50 nm. The number-average elementary particle diameter of said titanium dioxide particles may be determined by any known method in the art such as transmission electron microscopy (TEM).

According to a preferred embodiment of the invention, the titanium dioxide is coated. Uncoated titanium dioxide is photocatalytic and should be avoided in sunscreens. Preferably, the titanium dioxide is a coated titanium dioxide which is a pigment that has undergone one or more surface treatments of chemical, electronic, mechanochemical and/or mechanical nature preferably with compounds selected from the group consisting of amino acids, beeswax, fatty acids, fatty alcohols, anionic surfactants, lecithins, sodium, potassium, zinc, iron salts of fatty acids (stearic acid, metal alkoxides (of titanium), polyethylene, silicones (such as dimethicone, cyclomethicone, polysilicone, simethicone, dimethicone/methicone copolymer and/or cyclopentasiloxane), proteins (collagen, elastin), alkanolamines, silicon oxides, metal oxides, sodium hexametaphosphate, silica and mixtures thereof, more preferably wherein the titanium oxide is coated with stearic acid, silica, dimethicone and mixtures thereof.

Preferably, the coated titanium dioxide pigments are titanium oxides that have been coated:
with silica, such as the product Sunveil from the company Ikeda, the product Eusolex T-AVO from the company Merck, and the product Sunsil TIN 50 from the company Sunjin;
with silica and iron oxide, such as the product Sunveil F from the company Ikeda; and Microtitanium Dioxide MT 100 SA from the company Tayca and Tioveil from the company Croda;
with iron oxide and iron stearate, such as the product Microtitanium Dioxide MT 100 F from the company Tayca;
with silica and a silicone, such as the product UV-Titan X 195 from the company Sachtleben Pigments, and Parsol TX from the company DSM Nutritional Products;
with silica and cetyl phosphate such as the product Eusolex T-easy from Merck;
with UV Titan M 262 (with dimethicone) from the company Sachtleben Pigments; Eusolex T-2000 (with simethicone) from Merck;
with triethanolamine, such as the product STT-65-S from the company Titan Kogyo;
with stearic acid, such as the product Tipaque TTO-55 (C) from the company Ishihara;
with sodium hexametaphosphate, such as the product Microtitanium Dioxide MT 150 W from the company Tayca;
with silicone such as trimethoxycaprylylsilane or simethicone;
with octyltrimethylsilane, notably sold under the trade name T 805 by the company Degussa Silices;
with a polydimethylsiloxane, notably sold under the trade name 70250 Cardre UF TiO2SI3 by the company Cardre;
anatase/rutile TiO2 treated with a polydimethylhydrogenosiloxane, notably sold under the trade name Microtitanium Dioxide USP Grade Hydrophobie by the company Color Techniques.

Mention may also be made of titanium dioxide pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids.

According to one embodiment of the invention, the cosmetic composition comprises titanium dioxide in an amount of from 0.5 to 25.0 wt. %, or of from 0.5 to 20 wt. %, or of from 1.0 to 15.0 wt. %, or of from 1.5 to 10 wt. %, or of from 2.0 to 8.0 wt. %, or of from 4.0 to 7.0 wt. %, or of from 4.5 to 5.5 wt. %, based on the total weight of the cosmetic composition. The cosmetic composition described herein can, for example, comprise titanium dioxide in an amount of 0.5 wt. %, or of 1.0 wt. %, or of 1.5 wt. %, or of 2.0 wt. %, or of 2.5 wt. %, or of 3.0 wt. %, or of 3.5 wt. %, or of 4.0 wt. %, or of 4.5 wt. %, or of 5.0 wt. %, or of 5.5 wt. %, or of 6.0 wt. %, or of 6.5 wt. %, or of 7.0 wt. %, or of 7.5 wt. %, or of 8.0 wt. %, or of 8.5 wt. %, or of 9.0 wt. %, or of 9.5 wt. %, or of 10.0 wt. %, or of 10.5 wt. %, or of 11.0 wt. %, or of 11.5 wt. %, or of 12.0 wt. %, or of 12.5 wt. %, or of 13.0 wt. %, or of 13.5 wt. %, or of 14.0 wt. %, or of 14.5 wt. %, or of 15.0 wt. %, or of 15.5 wt. %, or of 16.0 wt. %, or of 16.5 wt. %, or of 17.0 wt. %, or of 17.5 wt. %, or of 18.0 wt. %, or of 18.5 wt. %, or of 19.0 wt. %, or of 19.5 wt. %, or of 20.0 wt. %, or of 20.5 wt. %, or of 21.0 wt. %, or of 21.5 wt. %, or of 22.0 wt. %, or of 22.5 wt. %, or of 23.0 wt. %, or of 23.5 wt. %, or of 24.0 wt. %, or of 24.5 wt. %, or of 25.0 wt. %, based on the total weight of the cosmetic composition.

According to one embodiment of the invention, the cosmetic composition comprises
(i) bemotrizinol (INCI: bis-ethylhexyloxyphenol methoxyphenyl triazine) in an amount of 0.5 to 6.0 wt. %;
(ii) Phenyl-3H-benzimidazole-5-sulfonic acid (Ensulizole) in an amount of 1.0 to 4.0 wt. %;
(iii) zinc oxide in an amount of 0.5 to 25.0 wt. %;
(iv) titanium oxide in an amount of 0.5 to 25.0 wt. %;
each based on the total weight of the cosmetic composition.

According to one embodiment of the invention, the cosmetic composition comprises
(i) bemotrizinol (INCI: bis-ethylhexyloxyphenol methoxyphenyl triazine) in an amount of 1.5 to 5.0 wt. %;
(ii) Phenyl-3H-benzimidazole-5-sulfonic acid (Ensulizole) in an amount of 1.5 to 3.0 wt. %;
(iii) zinc oxide in an amount of 2.5 to 10.0 wt. %;
(iv) titanium oxide in an amount of 2.5 to 10.0 wt. %;
(v) a neutralizing agent selected from tromethamine, triethanolamine, tetrahydroxypropyl ethylenediamine, arginine, and aminomethylpropanol in an amount of 0.5 to 4.0 wt. %,
each based on the total weight of the cosmetic composition.

According to one embodiment of the invention, the cosmetic composition comprises
(i) bemotrizinol (INCI: bis-ethylhexyloxyphenol methoxyphenyl triazine) in an amount of 2.0 to 5.0 wt. %;
(ii) Phenyl-3H-benzimidazole-5-sulfonic acid (Ensulizole) in an amount of 2.0 to 2.5 wt. %;
(iii) zinc oxide in an amount of 3.5 to 6.5 wt. %;
(iv) titanium oxide in an amount of 3.5 to 6.5 wt. %;
(v) a neutralizing agent selected from tromethamine, triethanolamine, tetrahydroxypropyl ethylenediamine, arginine, and aminomethylpropanol in an amount of 0.7 to 2.5 wt. %,
each based on the total weight of the cosmetic composition.

According to a preferred embodiment of the invention, the cosmetic composition is considered water resistant in accordance with the contact angle measurement method having a contact angle of more than 30°. According to one embodiment of the invention, the cosmetic composition exhibits a contact angle of at least 35°, using the contact angle measurement method. According to another embodiment, the cosmetic composition exhibits a contact angle of at least 40°, using the contact angle measurement method.

The contact angle measurement method refers to a method of determining the water resistance of a UV filter containing composition by using the wettability of a treated skin area. The method is described in detail in R. Hagens et al., “Contact angle measurement—a reliable supportive method for screening water-resistance of ultraviolet-protecting products in vivo”, International Journal of Cosmetic Science, 2007, 29, 283-291. It is based on the concept that the higher the wettability of a sunscreen-treated skin area, the higher is the wash-out rate of topically applied UV filter compounds. Details of the method are further disclosed in the experimental section.

The inventive cosmetic composition preferably comprises additives, in particular selected from the group consisting of emulsifiers, emollients, further viscosity regulators (thickeners), sensory enhancers, humectants, film formers, UV scattering compounds, adjuvants such as anti-aging actives, vitamins, antioxidants, chelating agents, preservatives, perfumes and combinations thereof.

Preferred O/W emulsifiers include, without limitation:
glucose derivatives such as cetearyl glucoside, arachidyl glucoside, lauryl glucoside, polyglyceryl-3 methylglucose distearate, methyl glucose sesquistearate,
sucrose derivative such as sucrose polystearate, sucrose palmitate,
sorbitol derivatives such as polysorbate derivatives, inulin derivatives such as inulin lauryl carbamate,
glycerides of fatty acids such as glyceryl stearate, glyceryl stearate SE, glyceryl stearate citrate,
glumatic acid derivatives such as sodium stearoyl glutamate,
sulfosuccinic acid derivatives such as disodium cetearyl sulfosuccinate,
phosphoric acid derivatives such as potassium cetyl phosphate, ceteh-10 phosphate, C20-22 alkyl phosphate,
fatty acid esters of polyglyceryl such as polyglyceryl-3-diisostearate, polyglyceryl-2-dipolyhydroxystearate, polyglyceryl-10 stearate,
oxyalkenylated fatty alcohol such as ceteareth-20, steareth-21, beneheth-25,
oxyalkenylated fatty acid such as PEG-100 stearate,
oxyalkenylated organomodified silicone/polysiloxane/polyalkyl/polyether copolymers and derivatives,
ethoxylated fatty acid soap,
phospholipids based such as lecithin derivatives.

Preferred emulsifiers to form a W/O emulsion include, without limitation:
glycerides of fatty acids such glyceryl oleate, sorbitan laurate,
sorbitan esters such as sorbitan oleate,
fatty acid esters of polyglyceryl such as polyglyceryl-3-diisostearate, polyglyceryl-2-dipolyhydroxystearate, polyglyceryl-4 isostearate,
oxyalkenylated fatty alcohol such steareth-2,
oxyalkenylated fatty acid such as PEG-30 dipolyhydroxystearate,
organomodified silicone/polysiloxane/polyalkyl/polyether copolymers and derivatives such as cetyl dimethicone copolyol, cetyl PEG/PPG-10/1 Dimethicone, PEG-10 dimethicone.

Preferred emollients include, without limitation:
esters of linear or branched fatty acids with linear or branched fatty alcohols such as propylheptyl caprylate, coco caprylate, isopropyl myristate, ethylhexyl palmitate,
esters of aromatic carboxylic acids with linear or branched fatty alcohols such as C₁₂-C₁₅-alkyl benzoate, ethylhexyl benzoate, phenethyl benzoate,
dicarboxylic acid esters with linear or branched alcohols such as dibutyl adipate, dicaprylyl carbonate, diisopropyl sebacate,
esters of hydroxycarboxylic acids with linear or branched fatty alcohols,
esters of linear or branched fatty acids with polyhydric alcohol such as butylene glycol dicaprylate/dicaprate,
mono-, di-, tri-glycerides based on C₆-C₁₈ fatty acids such as caprylic/capric triglycerides, coco glycerides,
guerbet alcohols such as octyldodecynol,
hydrocarbons such as hydrogenated polyisobutene, mineral oil, squalene, isohexadecane,
ethers such as dicaprylyl ether,
silicone derivatives (organomodified polysiloxanes) such as dimethylpolysiloxane, cyclic silicones.

Preferred lipophilic thickeners include, without limitation:
fatty alcohols such as cetyl alcohol, cetearyl alcohol, stearyl alcohol; behenyl alcohol,
fatty acids such as stearic acid; palmitic acid,
fatty acid esters such as myristyl stearate, pentaerythrityl distearate, cetyl palmitate; Tribehenin, dextrin palmitate,
waxes such as beeswax, carnauba wax, microcrystalline wax, ceresin, ozocerite; Oryza Sativa bran Wax, sunflower wax,
hydrogenated vegetable oil, hydrogenated castor oil, hydrogenated vegetable glycerides,
hydrogenated castor oil/sebacic acid copolymer,
silica based derivatives such as silica, silica dimethyl silylate.

Hydrophilic stabilizers/thickeners include, without limitation:
natural gums such as xanthan gum, tara gum, carrageenan,
silicate derivatives such as magnesium aluminium silicates,
cellulose derivatives such as hydroxypropyl cellulose, microcrystalline cellulose,
starch derivatives such as hydroxypropyl starch phosphate.

Hydrophilic stabilizers/thickeners include, without limitation:
natural gums such as xanthan gum, tara gum, carrageenan, guar gum, agar gum, alginates, gellan gum,
silicate derivatives such as magnesium aluminium silicates,
cellulose derivatives such as hydroxypropyl cellulose, microcrystalline cellulose,
starch derivatives such as hydroxypropyl starch phosphate,
polyacrylates or homopolymers of reticulated acrylic acids or polyacrylamides such as carbomers, acrylate copolymers, acrylate/C10-C30-alkyl acrylate crosspolymer, acrylate/beheneth-25 methacrylate copolymer.

Preferred sensory enhancers include, without limitation:
polyamide derivatives such as nylon-12,
polymethyl methacrylates,
silica,
mica,
polymethylsilsesquioxane,
starch derivatives such as aluminum starch octenylsuccinate,
dimethicone derivatives,
boron nitride,
HDI/trimethylol hexyllactone crosspolymer,
perlite.

Also included may be UV protection scattering compounds. These include, without limitation:
microcrystalline cellulose,
calcium carbonate,
hydroxyapatite,
silica,
hollow spheres such as styrene/acrylates copolymer spheres.

Further components may belong to the class of film formers and film forming enhancers. Such compounds include, without limitation:
hydrogenated dimer dilinoleyl/dimethylcarbonate copolymer,
diisostearoyl polyglyceryl-3 dimer dilinoleate,
polyglyceryl-3 stearate/sebacate crosspolymer,
cellulose derivatives, such as methylcellulose and ethylcellulose,
capryloyl glycerin/sebacic acid copolymer.

Also included may be UV protection booster compounds. These include, without limitation:
benzotriazolyl dodecyl p cresol,
ethylhexyl methoxycrylene,
polyester-8,
diethylhexyl syringylidenemalonate,
trimethoxybenzylidene pentanedione,
diethylhexyl 2,6-naphthalate,
Bis(Cyano Butylacetate) 10 Anthracenediylidene,
polyester-25,
butyloctyl salicylate.

Preferred humectants include, without limitation:
glycerin,
butylene glycol,
propylene glycol,
sorbitol,
beta-glucan,
1,2 pentadiol,
1,2 hexandiol,
1,2 octandiol,
1,2 decandiol,
2-Methyl-1,3-propandiol.

Preferred adjuvants include, without limitation:
tocopherol derivatives,
retinol derivatives,
ascorbic acid derivatives,
bisabolol,
allantoin,
panthenol,
chelating agents (EDTA, EDDS, EGTA, phytic acid, piroctone olamine),
ethylhexyl glycerin,
hydroxyacetophenone,
caprylhydroxymic acid,
propellants such as propane, butane, isobutene, dimethyl ether,
insect repellants such as butylacetylaminopropionate.

Preferred preservatives include, without limitation:
benzyl alcohol,
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

It is preferably that the inventive cosmetic composition comprises two or more of the additives described above. In connection with the above preferred embodiments, it is to be understood that if the cosmetic composition comprises two or more additives, combinations of any of the additives as defined above are also part of the invention.

In connection with the above preferred and particularly preferred embodiments, it is to be understood that the cosmetic composition may further comprise water.

In case water is present in the cosmetic composition it is to be understood that it is preferably present in an amount of more than 5.0% by weight, based on the total weight of the composition.

In one embodiment of the present invention, the cosmetic composition can be provided in different forms, e.g. gels, creams, oils, lotions, sticks, foams, pastes or in the form of a sprayable product. The inventive cosmetic composition may inter alia be formulated as a emulsion such as water-in-oil (W/O), oil-in-water (O/W), water-in-oil-in-water (W/0/W), oil-in-water-in-oil (O/W/O), or an oil monophase, or oil-alcohol mixture, or a hydrophilic monophase, or a vesicular dispersion of an ionic or nonionic amphiphilic lipid, or a gel, or a solid lipophilic stick, a solid hydrophilic stick or a powder, or an aerosol formulation.

According to a preferred embodiment of the invention, the cosmetic composition has a UVA-PF that is at least one third of the SPF. It is furthermore preferred that the cosmetic composition shows a critical wavelength of at least 370 nm, thus providing excellent protection along the entire harmful UV radiation range. It is further preferred that the cosmetic composition has a protection ratio UVA1/UV equal or superior to 0.7 after and irradiation of 4 MED (according to the FDA proposed order (OTC000008) issued Sep. 24, 2021).

The UVA-PF can exemplarily be determined by simulation calculation using BASF sunscreen simulator (https://sunscreensimulator.basf.com/) or in vivo or in vitro.

Preferably, the UVA-PF is determined by simulation calculation.

It is further preferred that the cosmetic composition has a SPF (sun protection factor) value of minimum 6 up to 80.

According to one embodiment of the invention, the cosmetic composition has a SPF of more than 15 or more than 30 or more than 50.

The SPF can exemplarily be determined by simulation calculation using BASF sunscreen simulator (https://sunscreensimulator.basf.com/) or in vivo or in vitro. A suitable in vitro method may be performed as follows:
SPF in vitro determination is based on the assessment of UV-transmittance through a thin film of sunscreen sample spread on roughened substrate—SB6 PMMA-plates (4-5 μm roughness, area 4.8×4.8 cm) from Helioscreen, Creil, France. The sunscreen formulation to be tested was applied on the PMMA plate with an amount of 1.2 mg/cm². Three plates and 5 measurements per plate were done per formulation. The formulation was distributed manually as equal as possible on the plate. The plates applied with formulation were stored 30 minutes at room temperature and in a dark place prior the measurement.

In vitro transmission measurement were performed from 290-400 nm with 1 nm steps with the Labsphere UV Transmittance Analyzer UV 2000S. The UV transmission spectrum was acquired, and the SPF in vitro values were calculated according to equation below (mean SPF of all 15 values (from the three plates and 5 measurements per plate))

$$SPF = \frac{{\sum }_{290 nm}^{400 nm} {s}_{er} ( λ ) \cdot S ( λ )}{{\sum }_{290 nm}^{400 nm} {s}_{er} ( λ ) \cdot S ( λ ) \cdot T ( λ )}$$

where, ser(λ) is the erythema action spectrum and S(λ) is the spectral irradiance received from the UV source and T(λ) is the in vitro measured light transmittance.

In a preferred embodiment of the present invention, the cosmetic composition provides a photostability of more than 80%, preferably of more than 85%, and in particular of more than 90%. Preferably, the cosmetic composition provides a photostability after 10 MED, 20 MED, and/or 50 MED (for skin phototype II), of more than 80%, preferably of more than 85%, and in particular of more than 90%. The photostability of the compositions is preferably determined by applying the respective composition on roughened quartz plates (2 μl/cm²) and then irradiating the plates using an Atlas CPS device with a certain minimal erythemal dose (MED) irradiation, preferably 50 MED irradiation. Then, the plates are rinsed off with tetrahydrofuran and the rinsing solution is analyzed via HPLC to determine the recovery of the UV filters. An alternative preferred method for determining the photostability of the composition comprises the irradiation of sunscreen-covered PMMA plates and evaluation of the difference between the absorbance spectrum before and after irradiation.

The second aspect of the invention relates to a method for increasing the water resistance of a cosmetic composition comprising bis-ethylhexyloxyphenol methoxyphenyl triazine (bemotrizinol), zinc oxide, and titanium dioxide comprising the step of adding a water-soluble organic UV filter or a salt thereof to the cosmetic composition. Surprisingly, the addition of a water-soluble organic UV filter or a salt thereof increases the water resistance of a cosmetic composition comprising bemotrizinol, zinc oxide, and titanium dioxide as UV filter combination.

According to one embodiment of the second aspect of the invention, the cosmetic composition is free of the UV filters from the group consisting of hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), polysilicone-15, and 2-hydroxy-4-methoxybenzophenone (INCI benzophenone-3).

The statements made with regard to the embodiments of the first aspect of the invention also apply in the same way to the second aspect of the invention.

The invention is further illustrated by the following examples.

### EXAMPLES

### Methods

### Water Resistance Contact Angle Method

The water resistance was determined using a contact angle measurement and is based on the method described in R. Hagens et al., “Contact angle measurement—a reliable supportive method for screening water-resistance of ultraviolet-protecting products in vivo”, International Journal of Cosmetic Science, 2007, 29, 283-291.

The method was performed in the following way: The sunscreen formulation was applied on roughened substrate—SB6 PMMA-plates at a dose of 1.2 mg/cm², followed by a drying time of 30 min. To determine the contact angle between water and the sunscreen-covered PMMA-plate, a drop of 8 μL of distilled water was placed on the sunscreen-covered PMMA-plate using an automatic application device (Easy Drop device from Kruss GmbH). The contour of the drop was recorded by an integrated Charge-Coupled Device (CCD) at a rate of two pictures per second during the first 5 s after placement of the water drop. The recorded pictures were subjected to automatic contour analysis using analysis software (Software DAS) which calculated the contact angle of all recorded pictures between a virtual baseline (=PMMA surface) and a virtual tangent (tangent 1 process of the used software) going through the contact point of the water surface with the plate. For each formulation, 5 independent water drops were measured on the same treated sunscreen covered PMMA plate, and the resulting average contact angle was calculated for each plate. Three plates were measured per formulation and the average contact angle of the three plates was calculated.

A sunscreen composition is considered to be water resistant if the contact angle is at least more than 30°.

### Formulations

The following five formulations were prepared:

TABLE 1
Formulations according to the invention (IF.1, IF.2, IF.3) and comparative
formulation CF.1; BEMT = Bis-Ethylhexyloxyphenol Methoxyphenyl
Triazine; PBSA = Phenylbenzimidazole Sulfonic Acid; all values
in wt. %, each based on the total weight of the formulation.

| | | Comp. | Inventive | Inventive | Inventive |
|---|---|---|---|---|---|
| | | Formulation | Formulation | Formulation | Formulation |
| Phase | INCI | CF.1 | IF.1 | IF.2 | IF.3 |
| A | Polyglyceryl-2- | 3.00 | 3.00 | 3.00 | 3.00 |
| | Dipolyhydroxystearate, | | | | |
| | Lauryl Glucoside, | | | | |
| | Glycerin | | | | |
| | Beheneth-25 | 1.00 | 1.00 | 1.00 | 1.00 |
| | Polyhydroxystearic Acid | 1.00 | 1.00 | 1.00 | 1.00 |
| | C12-15 Alkyl Benzoate | 10.00 | 10.00 | 10.00 | 10.00 |
| | Dibutyl Adipate | 10.00 | 10.00 | 10.00 | 10.00 |
| | Dicaprylyl Carbonate | 5.00 | 5.00 | 5.00 | 5.00 |
| | Phenoxyethanol and | 1.00 | 1.00 | 1.00 | 1.00 |
| | Ethylhexylglycerin | | | | |
| | Zinc Oxide (nano), | 4.00 | 3.50 | 3.50 | 4.00 |
| | Triethoxycaprylylsilane* | | | | |
| | Titanium Dioxide | 5.90 | 5.90 | 5.90 | 5.90 |
| | (nano), | | | | |
| | SilicaDimethicone** | | | | |
| | BEMT | 5.00 | 5.00 | 2.50 | 2.00 |
| | Avobenzone | — | — | 2.50 | — |
| B | Water | Qsp 100% | Qsp 100% | Qsp 100% | Qsp 100% |
| | Glycerin | 2.00 | 2.00 | 2.00 | 2.00 |
| | Xanthan Gum | 0.20 | 0.20 | 0.20 | 0.20 |
| | Disodium EDTA | 0.20 | 0.20 | 0.20 | 0.20 |
| C | Hydroxyethyl | 2.00 | 2.00 | 2.00 | 2.00 |
| | Acrylate/Sodium | | | | |
| | Acryloyldimethyl | | | | |
| | Taurate Copolymer | | | | |
| D | PBSA | — | 2.00 | 2.00 | 2.50 |
| | Water | — | 10.00 | 10.00 | 10.00 |
| | Tromethamine | — | 1.50 | 1.50 | 2.00 |

*Z Cote HP1 from BASF SE
**Parsol TX from DSM -Firmenich

The formulations were prepared by first heating phase A to 80° C. while stirring without Titanium dioxide (TiO2) and zinc oxide (ZnO). Then, at 80° C. TiO2 and ZnO were added under stirring into phase A and the mixture was homogenized with an ultra turrax type device. Phase B was heated to 80° C. under stirring. Phase A was added to phase B under stirring, then homogenized. Subsequently, ingredient C was added, and the mixture was homogenized. Finally, phase D which was previously prepared and homogenized was added to the mixture. The mixture was left to cool down to room temperature under stirring.

The water resistance by contact angle measurement method as described above was determined for each of the four formulations. The results are summarized in table 2:

| Formulation | CF.1 | IF.1 | IF.2 | IF.3 |
|---|---|---|---|---|
| Contact | 26° | 41° | 46° | 46° |
| angle | | | | |

As can be seen in table 2, the contact angle for the comparative formulation CF.1 was below the water resistance threshold of more than 30°, whereas the inventive formulations IF.1, IF.2 and IF.3 all had significantly larger contact angles of more than 40°. Therefore, the inventive formulations IF.1-IF.3 are all considered water resistant, whereas the comparative formulation without PBSA does not fulfil the water resistance requirement.

Therefore, the addition of a water-soluble organic UV filter such as phenylbenzimidazole sulfonic acid (Ensulizole) surprisingly increases the water resistance of a cosmetic composition comprising the UV filter combination of BEMT, and at least one inorganic UV filter, in particular a mixture of zinc oxide and titanium dioxide.

1. Cosmetic composition comprising
(i) bemotrizinol (INCI: bis-ethylhexyloxyphenol methoxyphenyl triazine),
(ii) a water-soluble organic UV filter or a salt thereof,
(iii) at least one inorganic UV filter,
wherein the cosmetic composition is free of the UV filters from the group consisting of hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), polysilicone-15, and 2-hydroxy-4-methoxybenzophenone (INCI benzophenone-3).

2. Cosmetic composition according to claim 1, wherein the cosmetic composition is free of 2-ethylhexyl-2-cyano-3,3-diphenylprop-2-enoate (INCI octocrylene).

3. Cosmetic composition according to claim 1, wherein the at least one inorganic UV filter (III) is selected from zinc oxide, titanium oxide, and mixtures thereof.

4. Cosmetic composition according to claim 1, wherein the cosmetic composition is free of (RS)-2-Ethylhexyl (2E)-3-(4-methoxyphenyl)prop-2-enoate (octinoxate).

5. Cosmetic composition according to claim 1, wherein the cosmetic composition is free of any further organic UV filter.

6. Cosmetic composition according to claim 1, wherein the water-soluble organic UV filter (ii) is selected from the group consisting of Phenyl-3H-benzimidazole-5-sulfonic acid (Ensulizole), [(3Z)-3-[[4-[(Z)-[7,7-Dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (Ecamsule), disodium phenyl dibenzimidazole tetrasulfonate (Bisdisulizole disodium), or their salts, and mixtures thereof.

7. Cosmetic composition according to claim 6, wherein the water-soluble organic UV filter (ii) is Phenyl-3H-benzimidazole-5-sulfonic acid (Ensulizole), or a salt thereof, and mixtures thereof.

8. Cosmetic composition according to claim 1, wherein the cosmetic composition further comprises an organic neutralizing agent.

9. Cosmetic composition according to claim 8, wherein the organic neutralizing agent is selected from tromethamine, triethanolamine, tetrahydroxypropyl ethylenediamine, arginine, and aminomethylpropanol.

10. Cosmetic composition according to claim 1, wherein the cosmetic composition comprises a Hydroxyethyl Acrylate/Sodium Acryloyldimethyl Taurate Copolymer.

11. Cosmetic composition according to claim 1, wherein the cosmetic composition comprises
(i) bemotrizinol (INCI: bis-ethylhexyloxyphenol methoxyphenyl triazine) in an amount of 0.5 to 6.0 wt. %;
(ii) Phenyl-3H-benzimidazole-5-sulfonic acid (Ensulizole) in an amount of 1.0 to 4.0 wt. %;
(iii) zinc oxide in an amount of 0.5 to 25.0 wt. %;
(iv) titanium oxide in an amount of 0.5 to 25.0 wt. %;
each based on the total weight of the cosmetic composition.

12. Cosmetic composition according to claim 1, wherein the cosmetic composition comprises
(i) bemotrizinol (INCI: bis-ethylhexyloxyphenol methoxyphenyl triazine) in an amount of 1.5 to 5.0 wt. %;
(ii) Phenyl-3H-benzimidazole-5-sulfonic acid (Ensulizole) in an amount of 1.5 to 3.0 wt. %;
(iii) zinc oxide in an amount of 2.5 to 10.0 wt. %;
(iv) titanium oxide in an amount of 2.5 to 10.0 wt. %;
(v) a neutralizing agent selected from tromethamine, triethanolamine, tetrahydroxypropyl ethylenediamine, arginine, and aminomethylpropanol in an amount of 0.5 to 4.0 wt. %,
each based on the total weight of the cosmetic composition.

13. Cosmetic composition according to claim 1, wherein the cosmetic composition comprises
(i) bemotrizinol (INCI: bis-ethylhexyloxyphenol methoxyphenyl triazine) in an amount of 2.0 to 5.0 wt. %;
(ii) Phenyl-3H-benzimidazole-5-sulfonic acid (Ensulizole) in an amount of 2.0 to 2.5 wt. %;
(iii) zinc oxide in an amount of 3.5 to 6.5 wt. %;
(iv) titanium oxide in an amount of 3.5 to 6.5 wt. %;
(v) a neutralizing agent selected from tromethamine, triethanolamine, tetrahydroxypropyl ethylenediamine, arginine, and aminomethylpropanol in an amount of 0.7 to 2.5 wt. %,
each based on the total weight of the cosmetic composition.

14. Cosmetic composition according to claim 1, wherein the cosmetic composition is considered water resistant in accordance with the contact angle measurement method having a contact angle of more than 30°.

15. Cosmetic composition according to claim 1, wherein the cosmetic composition has a SPF of more than 15 or more than 30 or more than 50 and/or wherein the cosmetic composition has a contact angle of more than 30° and/or wherein the cosmetic composition has a photostability of more than 80%.

16. Method for increasing the water resistance of a cosmetic composition comprising bis-ethylhexyloxyphenol methoxyphenyl triazine (bemotrizinol), zinc oxide, and titanium dioxide comprising the step of adding a water-soluble organic UV filter or a salt thereof to the cosmetic composition.
